(19) United States
(12) Patent Application Publication
(10) Pub. No.: US 2003/0114851 A1
(43) Pub. Date: Jun. 19, 2003

(54) Electrosurgical jaws for controlled application of clamping pressure

(76) Inventors: Csaba Truckai, Saratoga, CA (US); John H. Shadduck, Tiburon, CA (US)

Correspondence Address: CSABA TRUCKAI, 2629 B TERMINAL BLVD., MOUNTAIN VIEW, CA, 94043, US

(21) Appl. No.: 10/017,452

(22) Filed: Dec. 13, 2001

(51) Int. Cl. A61B 18/18

(52) U.S. Cl. 606/51; 606/205; 606/50

(57) ABSTRACT

An electrosurgical medical device and technique for creating thermal welds in engaged tissue that provides very high compressive forces. The working end comprises five basic components, including (i) a handle portion coupled to an introducer sleeve member that carries paired first and second jaws members at it distal end; (ii) an actuatable elongate member that transitions distally to first and second extension portions that carry respective first and second slidable cam-type surfaces for engaging the paired jaws; (iii) a transverse member that is connected to the first and second extension portions for adjusting the transverse dimension between the first and second slidable cam surfaces to thereby control clamping pressure; (iv) a mechanism in the handle for actuating the elongate member to open and close the jaws; and (v) a mechanism in the handle for adjusting the transverse dimension between the first and second cam surfaces via the transverse member.

### CROSS-REFERENCE TO RELATED APPLICATION

This application claims priority from U.S. Provisional Patent Application No. ______, filed Dec. 14, 2000 (Docket No. SRX-002) titled Electrosurgical Jaws for Controlled Application of Clamping Pressure which is incorporated herein by this reference. This application is also related to co-pending U.S. patent application Ser. No. ______ filed Oct. 23, 2000 (Docket No. SRX-001) titled Electrosurgical Systems and Techniques for Sealing Tissue which is incorporated herein by this reference.

### FIELD OF THE INVENTION

This invention relates to medical devices and techniques and more particularly relates to an electrosurgical jaw structure that allows substantially elongate jaws to apply controllable high compressive forces on the captured tissue volume for thermally sealing or welding the tissue volume, together with novel electrode arrangements for delivering Rf energy to the captured tissue volume.

### BACKGROUND OF THE INVENTION

In various open and laparoscopic surgeries, it is often necessary to seal or weld tissue volumes and thereafter transect the tissue, for example, in welding and transecting a blood vessel. In a typical procedure, a deformable metal clip may be used seal a blood vessel, or a stapling instrument may be used to apply a series of mechanically deformable staples to seal the edge a larger tissue volume that contains blood vessels. Such mechanical sealing devices can create a seal that leaks resulting in later complications.

Various radiofrequency (Rf) surgical instruments for sealing tissue volumes have been developed. For example, FIG. 1A shows a sectional view of paired electrode-jaws 2a and 2b of a typical prior art bi-polar Rf grasper that is grasping a blood vessel. In a typical bi-polar jaw arrangement, each jaw face comprises an electrode and Rf current flows across the tissue between the first and second polarities in the opposing jaws that engage opposing exterior surfaces of the tissue. FIG. 1A shows typical lines of bi-polar current flow between the jaws. Each jaw in FIG. 1A typically has a central slot adapted to receive a reciprocating blade member as is known in the art for transecting the captured vessel after it is sealed.

While bi-polar graspers as in FIG. 1A can adequately seal or weld tissue volumes that have a small cross-section, such bi-polar instruments are often ineffective in sealing or welding many types of anatomic structures, e.g., (i) substantially thick structures, (ii) large diameter blood vessels having walls with thick fascia layers f (see FIG. 1A), (iii) bundles of disparate anatomic structures, or (iv) structures having walls with irregular fibrous content.

As depicted in FIG. 1A, a relatively large diameter blood vessel falls into a category that is difficult to effectively weld utilizing prior art instruments. A large blood vessel wall has substantially thick, dense and non-uniform fascia layers underlying its exterior surface. As depicted in FIG. 1A, the fascia layers f prevent a uniform flow of current from the first exterior surface s to the second exterior surface s of the vessel that are in contact with electrodes 2a and 2b. The lack of uniform bi-polar current across the fascia layers f causes non-uniform thermal effects that typically result in localized tissue desiccation and charring indicated at c. Such tissue charring can elevate impedance levels in the captured tissue so that current flow across the tissue is terminated altogether. FIG. 1B depicts an exemplary result of attempting to weld across a vessel with thick fascia layers f with a prior art bi-polar instrument. FIGS. 1A-1B show localized surface charring c and non-uniform weld regions w in the medial layers m of vessel. Further, FIG. 1B depicts a common undesirable characteristic of prior art welding wherein thermal effects propagate laterally from the targeted tissue causing unwanted collateral (thermal) damage indicated at d.

A number of bi-polar jawed instruments adapted for welding and transecting substantially small structures have been disclosed, for example: U.S. Pat. No. 5,735,848 to Yates et al.; U.S. Pat. No. 5,876,410 to Schulze et al.; and U.S. Pat. No. 5,833,690 to Yates et al. One other similar bi-polar instrument was disclosed by Yates et al. in U.S. Pat. No. 5,403,312. In that patent, paired bi-polar electrodes are provided in left and right portions of a jaw member to induce current flow therebetween. It is not known whether a jaw having the left-to-right or side-to-side bipolar current flow of U.S. Pat. No. 5,403,312 was ever tested, but it seems likely that such an instrument would confine current flow to the tissue's exterior surface and facial layers f (see FIG. 1B), thus aggravating the desiccation and charring of such surface layers.

What is needed is an instrument working end for endoscopic procedures that can utilize Rf energy in new delivery modalities: (i) to weld or seal substantially thick anatomic structures; (ii) to weld or seal a blood vessel having non-uniform or thick fascia layers; (iii) to weld or seal tissue volumes that are not uniform in hydration, density and collagenous content; (iv) to weld a transected margin of a bundle of disparate anatomic structures; and (v) to weld a targeted tissue region while substantially preventing collateral thermal damage in regions lateral to the targeted tissue.

### SUMMARY OF THE INVENTION

The object of the present invention is to provide apparatus and techniques for causing controlled Rf energy delivery and controlled thermal effects within tissues having thick facial layers, or other tissue volumes with non-uniform fibrous content. For example, larger diameter blood vessels are a targeted application of the present invention since such vessels have thick facials layers that can prevent uniform current flow and uniform ohmic heating of the tissue.

In an exemplary embodiment, the working end of the electrosurgical instrument comprises five basic components, including (i) a handle portion coupled to an introducer sleeve member that carries paired first and second jaws members at it distal end; (ii) an actuatable elongate member that transitions distally to first and second extension portions that carry respective first and second slidable cam-type surfaces for engaging the paired jaws; (iii) a transverse member that is connected to the first and second extension portions for adjusting the transverse dimension between the first and second slidable cam surfaces to thereby control clamping pressure; (iv) a mechanism in the handle for actuating the elongate member to open and close the jaws; and (v) a mechanism in the handle for adjusting the transverse dimension between the first and second cam surfaces via the transverse member.

As background, the biological mechanisms underlying tissue fusion by means of thermal effects are not fully understood. In general, the delivery of Rf energy to a captured tissue volume causes ohmic resistive heating of tissue wherein the temperature thereby at least partially denatures tissue proteins. The objective is to denature such proteins, including collagen, into a proteinaceous amalgam that intermixes and fuses together as the proteins renature, thus causing an immediate seal. As the treated region heals over time, the so-called weld is reabsorbed by the body's wound healing process.

In order to create an effective weld in a tissue volume with substantial fascial layers, it has been found that several factors are critical. The objective is to create a substantially even temperature distribution across the targeted tissue volume to thereby create a uniform weld or seal. Fibrous tissue layers (i.e., fascia) conduct Rf current differently than adjacent less-fibrous layers, and it is believed that differences in extracellular fluid contents in such adjacent tissues contribute greatly to the differences in electrical resistance. It has been found that by applying very high compressive forces to a tissue volume comprising fascia layers and adjacent non-fibrous layers, the extracellular fluids either migrate from the non-fascial layers to the fascial layers or migrate from the captured tissue to collateral regions. In either event, high compressive forces tend to make the resistance more uniform regionally within the captured tissue volume. Further, it is believed that high compressive forces (i) cause protein denaturation at a lower temperature which is desirable, and (ii) cause enhanced intermixing of denatured proteins thereby creating a more effective weld upon tissue protein renaturation.

Also equal importance, it has been found that that a critical factor in creating an effective weld across adjacent fibrous (fascia) layers and non-fibrous (medial) layers is the deliver of bi-polar Rf energy from electrode surfaces having a very large surface area for engaging the maximum amount of the tissue surface. Further, it has been found that it is important to rapidly alternate current flow through the engaged tissue between parallel to the tissue engagement plane and orthogonal to the engagement plane to thereby accomplish uniform tissue heating and to avoid surface desiccation.

More in particular, the working end of the instrument carries a jaw assembly with paired first and second jaws for engaging and compressing tissue within an engagement plane. In a preferred embodiment, the jaw structure is moved between an open position and closed position about the tissue engagement plane by a dual-action jaw closing system: a low-compression jaw closing mechanism for typical grasping purposes and sealing purposes, and a high-compression jaw closing mechanism for applying high compressive forces to elongate jaws.

Of particular interest, the high-compression jaw closing mechanism provides for adjustability of the clamping or compressive forces applied to tissue captured between the paired jaws. In a preferred embodiment, the high-compression jaw closing mechanism utilizes an axially extending member that carries first and second cam surfaces that slidably engage cooperating jaw surfaces. The mechanism is novel in its use of a wire element that has an adjustable free length to thereby vary the transverse dimension between the cam surfaces-thereby providing for adjustable compressive forces applied by the jaws. Further, the wire element serves as a cutting electrode. The jaw structure also provides a floating pivot for the rotation of the jaws to allow the jaw faces to be parallel no matter the thickness of the engaged tissue.

The jaw structure in accordance with the invention further provides electrodes with greatly increased surface areas relative to the cross-section of the working end for accomplishing the electrosurgical welding method of the invention. The jaw faces that carry the electrode arrangement have cooperating undulating or angled shapes, rather than shaped as a radial of the jaw axis, to thereby increase the surface are in contact with tissue.

Further, the opposing jaws carry spaced apart electrodes surfaces in left and right sides of the jaws that are each coupled to an electrical source and controller to allow alternation of current flow in two manners: parallel to the engagement plane and orthogonal to the engagement plane. It has been found that by rapidly alternating current flow in the two manners (parallel and orthogonal to engagement plane) that uniform heating of thick or non-uniform fascia layers can be accomplished.

In another embodiment of the invention, the jaw assembly further includes components of a sensor system that together with a power controller can control Rf energy delivery during a tissue welding procedure. For example, feedback circuitry for measuring temperatures at one or more temperature sensors in the jaws may be provided. Another type of feedback circuitry may be provided for measuring the impedance of tissue engaged between the transecting member and a jaw. The power controller may continuously modulate and control Rf delivery in order to achieve (or maintain) a particular parameter such as a particular temperature in tissue, an average of temperatures measured among multiple sensors, a temperature profile (change in energy delivery over time), or a particular impedance level or range.

Additional objects and advantages of the invention will be apparent from the following description, the accompanying drawings and the appended claims.

### BRIEF DESCRIPTION OF THE DRAWINGS

FIG. 1A is an illustration of current flow between the paired jaws of a prior art bi-polar radiofrequency device in a method of sealing a blood vessel having substantially thick fascia layers.

FIG. 1B illustrates representative weld effects of the bi-polar current flow of FIG. 1A.

FIG. 2 is a perspective cut-away view of a Type “A” electrosurgical working end in accordance with the present invention showing the cooperating jaw members in a first (open) position having dual jaw closing mechanisms.

FIG. 3 is a different perspective cut-away view of the working end of FIG. 2 with the jaw members actuated to a second (closed) position by a first jaws closing mechanism; the second jaw closing mechanism comprising an axially extendable member still in a first (retracted) position.

FIG. 4 is a perspective view of the slidable blade member of the working end of FIGS. 2-3 in first and second positions with the jaw members not shown.

FIG. 5 is a perspective view of the axially extendable member of the working end of FIGS. 2 & 3 that comprises the second jaw closing mechanism in a second (extended) position, with the jaws themselves not shown for purposes of explanation.

FIG. 6 is another perspective view of the axially extendable member of FIG. 5 this time showing its proximal end within a handle portion of the instrument, the axially extendable member again in the second (extended) position with a phantom view of a jaw portion in thus in the second (closed) position.

FIG. 7 is another perspective view of the axially extendable member of FIGS. 5-6 in its second (extended) showing the adjustability of the transverse dimension between first and second cam surfaces of the extendable member.

FIG. 8 is a cross-sectional view of exemplary jaw faces of FIGS. 2-3 taken along line 8-8 of FIG. 3 showing the novel tissue engagement plane of the jaw structure with tissue captured within the jaws.

FIG. 9 is a cross-sectional view of the tissue engagement plane of a typical prior art electrosurgical jaw wherein the jaw faces extend generally as a radial of the central axis.

FIG. 10A is a sectional graphical view of an alternative preferred jaw shape (similar to FIG. 8) that (i) defines an engagement plane for providing increased electrode surface area; (ii) provides a graphical view of a blood vessel captured in the engagement plane under low compressive forces in accordance with the invention; and (iii) provides a graphical view illustrating a first method of the invention wherein the electrical source provides vectors of electrical current flow that parallel the engagement plane.

FIG. 10B is a graphical view of the jaws of FIG. 10A that (i) illustrated the transection of the captured tissue with the translatable transverse wire electrode; (ii) provides a graphical view of the captured blood vessel captured under very high compressive forces in accordance with the invention; and (iii) provides a graphical view of a second method of the invention wherein the electrical source provides vectors of electrical current flow that are orthogonal to the engagement plane.

FIG. 11 is a perspective view of a translatable component of a Type “B” embodiment of electrosurgical working end with cam surfaces carried at the ends of springable extension arms.

FIG. 12A is a perspective view of a translatable component of a Type “C electrosurgical working end with cam surfaces that have an adjustable spaced apart transverse dimension.

FIG. 12B is a perspective view of the de-mated transverse member of FIG. 12A.

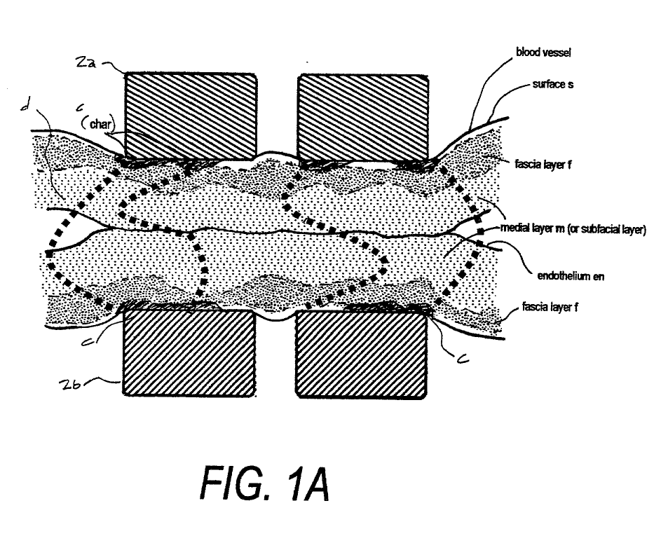

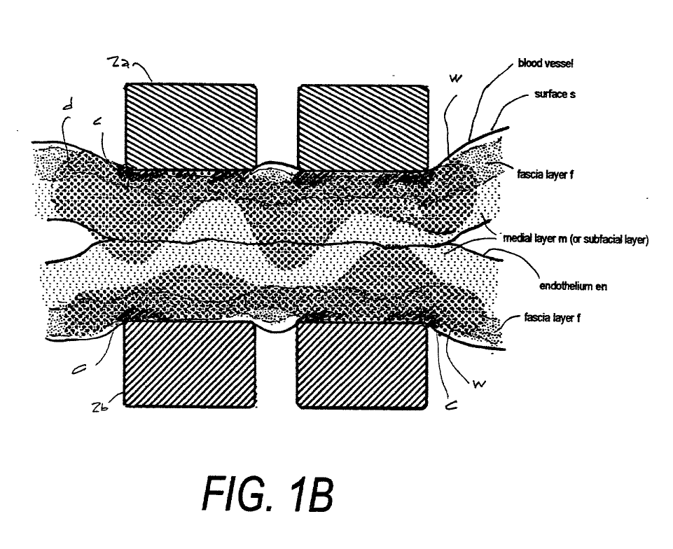

### DETAILED DESCRIPTION OF THE INVENTION

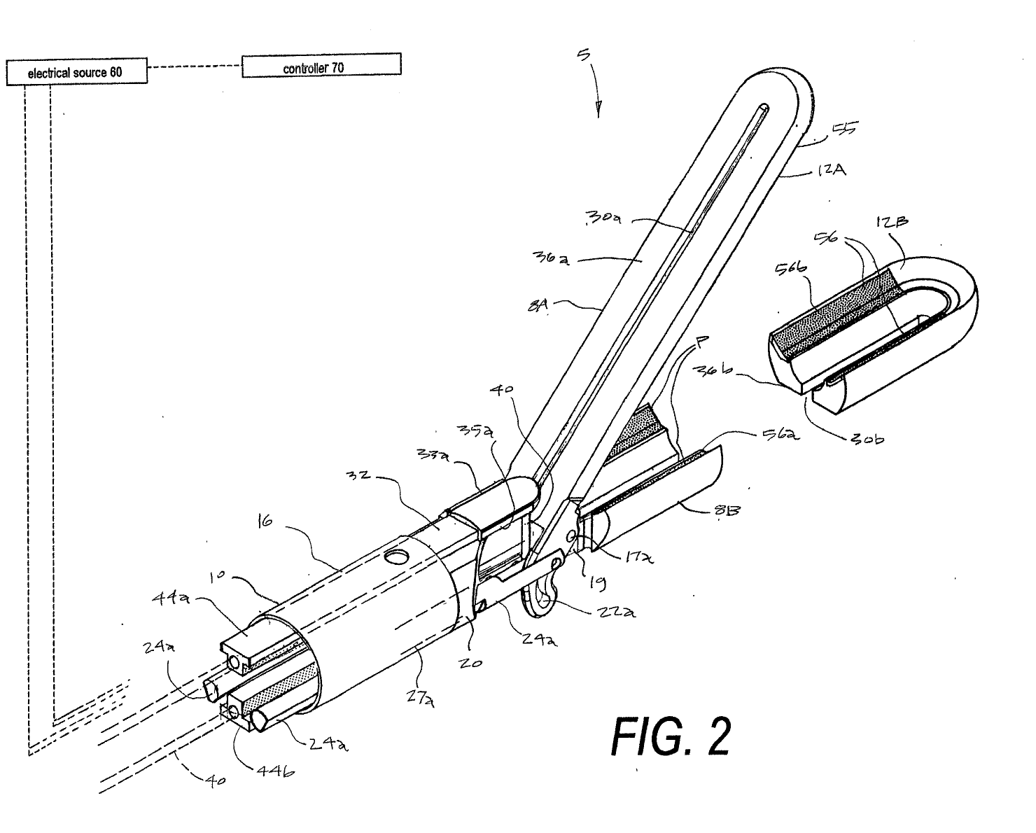

1. Type “A” Electrosurgical Jaw Structure for Sealing or Welding Tissue. Referring to FIG. 2, the working end 5 of an exemplary Type “A” embodiment is shown that is adapted for sealing or welding a tissue volume, such as a blood vessel, in an endoscopic procedure. The system provides apparatus that fall into two fields—which in i combination allow for creating effective welds or seals in tissue over the length of an elongate jaw. First, it has been found that an effective electrosurgical weld in thick or non-uniform tissues can be created if sufficient compressive forces are created by the jaw structure of the instrument. Therefore, the invention provides mechanisms for controllably creating high compressive forces over elongate jaws. Second, it has been found that particular manners of delivering energy from bi-polar electrode arrangements within specially shaped engagement surfaces are needed to weld large tissue volumes. These two aspects of the invention will be described in order below: first, relating to high compression jaw closure; and, secondly to novel electrode arrangements.

a. Controllable compression jaw closing mechanisms. Referring to FIGS. 2 & 3, the working end 5 has paired jaw portions 8A and 8B that are carried at the distal end of elongate introducer sleeve member 10 (with handle portion not shown) extending along central longitudinal axis 15. In this exemplary embodiment, the paired jaws have respective tissue-engaging surfaces 12A and 12B that have particular cooperating shapes-together with electrodes—that will be described further below. The structural component of the invention comprising the introducer member 10 has a cylindrical cross-section and comprises a tubular sleeve 16 that extends from the proximal handle. The diameter of sleeve 16 may range from about 3 mm. to 10 mm., e.g., to cooperate with a standard endoscopic trocar sleeve. The handle may be any type of pistol-grip or other type of handle known in the art that carries actuator levers, triggers or sliders known in the art and need not be described in any detail.

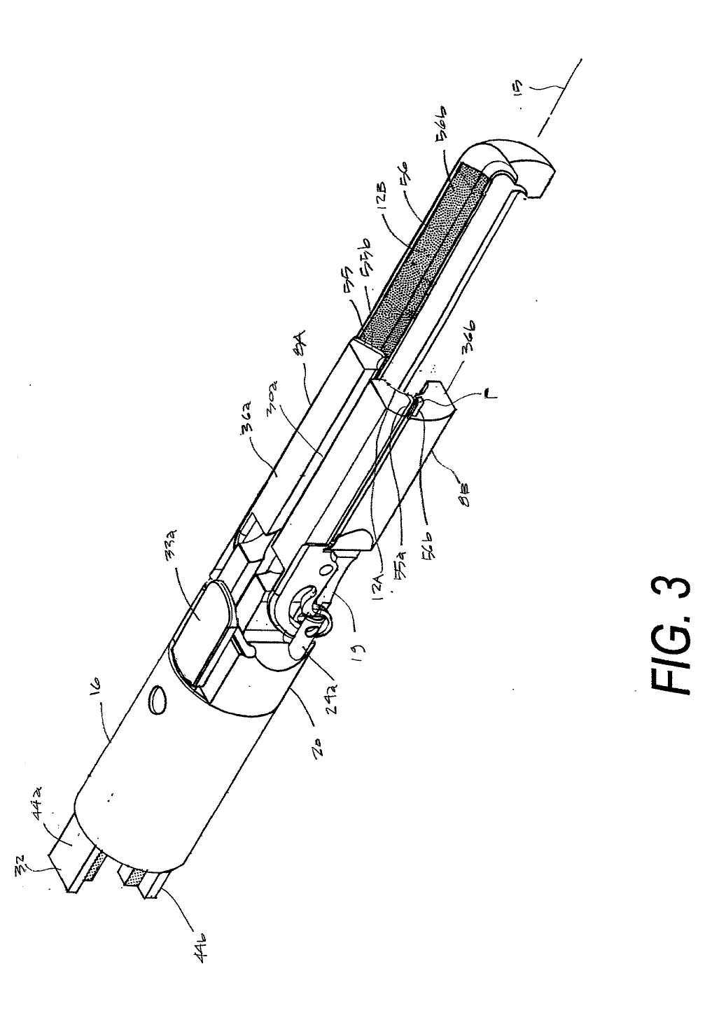

In this embodiment, the jaw structure is moved from the first open position (FIG. 2) to the second closed position (FIG. 3) about a tissue-engagement plane p by a dual-action jaw closing system. The jaw closing mechanisms can apply either low compression forces or very high compressive forces over the length of substantially elongate jaws. The first jaw closing mechanism is adapted for typical grasping purposes and also for initially sealing or cauterizing tissue with the electrode arrangement described later. It also may be used for sealing thin tissues. For reasons described herein, this first mechanism is called a low-compression jaw closing mechanism wherein the second mechanism is called a high-compression jaw closing mechanism for making elongate welds in tissue.

FIGS. 2 & 3 show the actuation of the low-compression jaw closing mechanism wherein the first jaw 8A is actuated from the first open position of FIG. 2 to the second closed position of FIG. 3. Referring to FIG. 2, it can be seen that the upper (first) jaw member 8A is actuatable about pivot pins 17a and 17b to open and close relative to the lower (second) jaw member 8B that in this exemplary embodiment is configured as a fixed (non-actuatable) jaw. The pivot pins 17a and 17b are fit into a proximal portion 19 of lower jaw body member 20 that is fixedly coupled to the distal end of introducer sleeve member 16.

In FIG. 2, it can be seen that upper jaw member 8A has arcuate slots 22a and 22b in arm portions 23a and 23b. Reciprocatable actuator rods 24a and 24b carry pins 26a and 26b in their distal ends that slide in arcuate slots 22a and 22b of the jaw arms to actuate upper jaw 8A from the open position of FIG. 2 toward a closed position of FIG. 3. Thus, it can be seen that the slidable actuator rods 24a and 24b comprise the first mechanism for rapidly closing the jaws—fro applying low to medium compression closing forces on the jaws. The proximal ends of actuator rods 24a and 24b are coupled to a first actuator known in the art and carried in the handle portion (e.g., a lever arm, squeeze grip, trigger or sliding actuator) that translates the rods to and fro. These rods 24a and 24b slide through parallel alignment bores 27a and 27b in body member 20.

The axial length of jaws 8A and 8B indicated at a may be any suitable length depending on the anatomic structure targeted for transection and sealing and may range from about 200 mm. or more, for example for resecting and sealing lung, although the scope of the invention covers jaws for a micro-surgery having a length of as little as 5.0 mm.

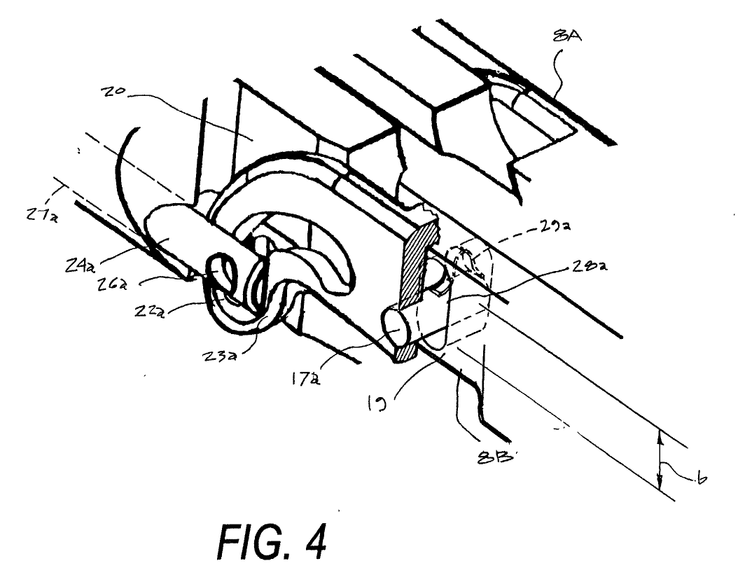

Referring now to FIG. 4, the pivot pins 17a and 17b fixed in upper jaw 8A that pivotably couple to jaw to the instrument body are preferably (but optionally) fit into elongate vertical slots 28a and 28b on either side of proximal portion 19 of body member 20 that is carried in the distal end of introducer sleeve member 16. A strong leaf spring 29a maintains the pivot pin 17a in a lower position in slot 28a thereby establishing the actual pivot point. It can be easily understood from FIG. 4 that closing the jaw structure on a thick tissue volume can overcome the strength of spring 29a to thereby move the actual pivot point vertically in slot 28a over a range indicated at b. As will be described below, the advantage of such a vertically moveable pivot point is that the jaws 8A and 8B can be closed over a substantially thick tissue volume captured within tissue-engaging plane p while at the same time the jaws faces 12A and 12B can remain spaced apart in a parallel orientation. In a dynamic pivot location were not provided, the distal end of the jaws faces could be spaced further apart than the proximal end of the jaw faces and an electrosurgical weld might not be as effective. This advantage will be described further below in the method of the invention. The pivot pin 17a is biased toward the lower position in slot 28a in FIG. 4 by a leaf spring, but any type of spring is acceptable. The vertical dimension of slots 28a and 28b are any suitable dimension, which together with the strength of springs 29a and 29b, to cooperate with the second jaw closing to clamp any selected thickness of tissue.

Referring to FIGS. 2, 3 & 5, the high-compression jaw closing mechanism can now be described. As best seen in FIG. 2, the upper and lower jaws 8A and 8B have axial slots therein indicated at 30a and 30b that cooperate to receive an axially extendable member 32 that carries distal body portions 33a and 33b that define first and second cam surfaces 35a and 35b for slidably contacting cooperating surfaces 36a and 36b of the first and second jaws 8A and 8B. The axially extendable member 32 can move distally and proximally by actuation of a lever or other means in the handle of the device (not shown).

The cooperating cam surfaces 35a and 35b define a transverse dimension d between the paired jaw's tissue-engaging faces 12A and 12B (the term transverse dimension d as used herein refers to either the dimension between the jaws faces 12A and 12B or the dimension between the cam surfaces 35a and 35b since they correspond to one another). Of particular interest, the invention provides a mechanism that allows the operator (i) to adjust the transverse dimension d between the first and second cam surfaces 35a and 35b between set dimensions, or (ii) to allow for dynamic adjustment of the transverse dimension d in response to tissue volume captured between the paired jaws. More in particular, a transverse member 40 comprising a wire element is provided that couples together the first and second cam surfaces 35a and 35b.

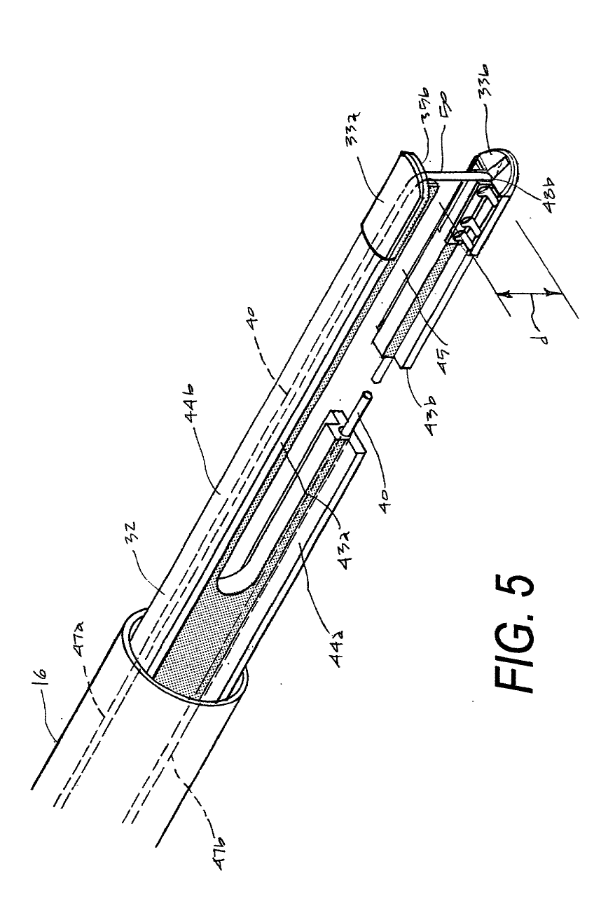

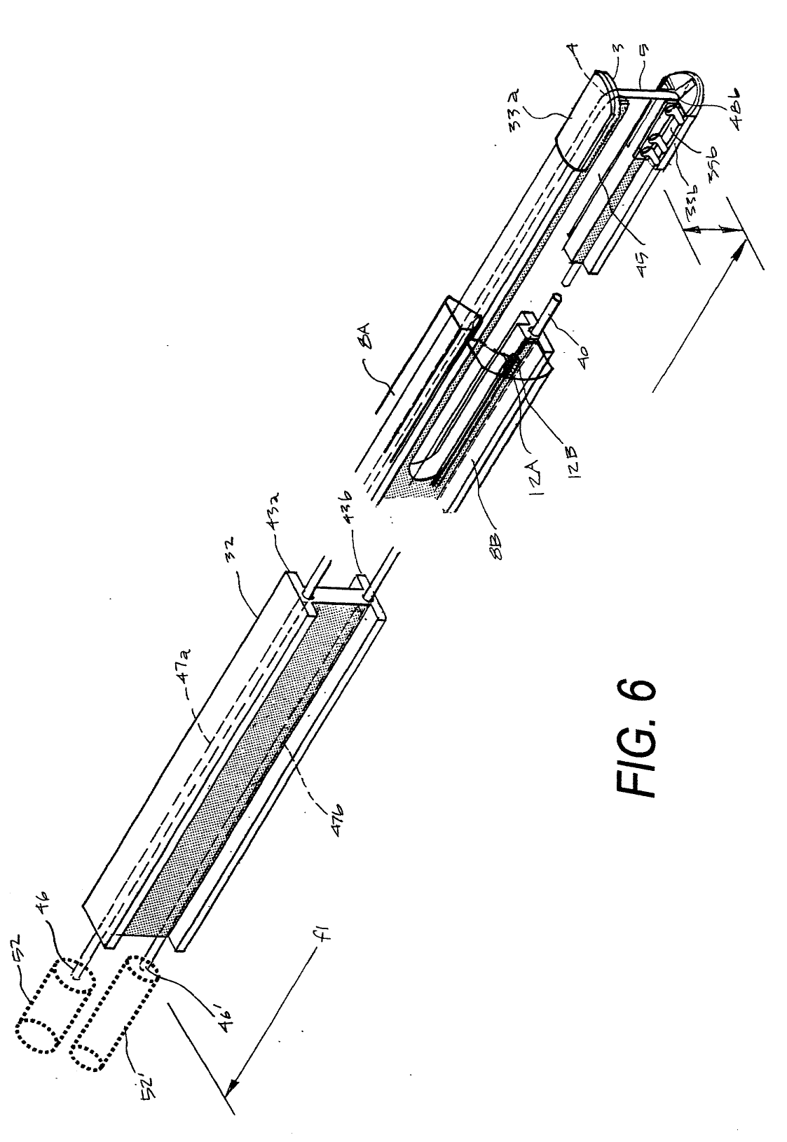

To more easily explain the operation of the axially extendable member 32 for controllably applying compression forces on the jaws, FIG. 5 shows introducer member 16 with the axially extendable member 32 in plain view without showing the jaws 8A and 8B. FIG. 5 shows axially extendable member 32 in its second extended position relative to the introducer 16, which can be compared with FIGS. 2 & 3 wherein the extendable member 32 is in its first retracted position. FIG. 6 shows this exemplary embodiment of extendable 32 de-mated entirely from the instrument. The proximal portion 41 of extendable member 32 has an “I”-beam type cross-section 42 with upper flange portion 43a and lower flange member 43b. The extendable member 32 is of a plastic or metal material that is somewhat flexible for reasons described below.

Of particular interest, the distal portion of the axially extendable member 32 transitions into first (upper) and second (lower) extension members or arms 44a and 44b that are spaced apart by intermediate space 45. As can be seen in FIGS. 5 & 6, these extension arms 44a and 44b carry the first (upper) and second (lower) cam surfaces 35a and 35b (in body portions 33a and 33b) that are spaced apart by transverse dimension d.

It can be easily seen in FIGS. 5 & 6 how the transverse wire member 40 is adjustable to control the transverse dimension d between the first and second cam surfaces 35a and 35b to thereby provide greater or lesser compressive force on tissue captured in the tissue-engaging plane p between jaws 8A and 8B. The assembly comprising the axially extendable member 32 and transverse wire member 40 of FIG. 6 are slidably carried in introducer member 16 (see FIG. 5) such that the entire assembly can be actuated forward (distally) and rearward (proximally). The transverse wire member 40 has a free length fl that extends between a first end 46 of the wire and a second end 46′ of the wire. FIG. 6 shows that wire member 40 extends from its first end 46 in a first bore 47a in extendable member 32 to then exit an opening 48a in upper cam surface 35a. Thereafter, the flexible wire 40 bends 90° to define a transverse wire portion 50 that extends across space 45 between the distal ends of extension arms 44a and 44b and the first and second cam surfaces 35a and 35b. The flexible wire member 40 then enters opening 48b in lower cam surface 35b and bends 90° to travel through second bore 47b in extendable member 32 to its second end indicated at 46′.

Still referring to FIG. 6, it can be understood that by adjusting the free length fl of the wire member 40, the transverse dimension d between the first and second cam surfaces 35a and 35b can be adjusted, assuming that forces are in play to open space 45 between cam surfaces 35a and 35b as would be the case wherein tissue was be captured between jaws 8A and 8B that resists compression. The free length fl of wire member 40 can adjusted in two ways, both of which are depicted schematically in FIG. 6: (i) the free length fl of the wire 40 can be dynamically adjustable under loading from the resistance to compression of tissue; or (ii) the free length fl of the wire 40 can be fixed at any particular selected length to provide a selected transverse dimension d between the jaw's tissue-engaging faces 12A and 12B no matter the thickness of the tissue.

To provide a dynamic or responsive adjustability to the transverse dimension d between the jaw's tissue-engaging faces 12A and 12B, FIG. 6 shows that first end 46 of wire member 40 is coupled to a spring tensioner mechanism indicated at 52. The spring tension mechanism can be a coil spring or any other type of spring. In this example, the second end 46′ of wire member 40 is fixedly coupled to extendable member 32.

FIG. 6 further shows the inventive means for providing any adjustable selected length to wire member 40 to provide a selected transverse dimension d between the jaw's tissue-engaging faces 12A and 12B. In this case, the second end 46′ of wire member 40 is coupled to a wire length adjustment mechanism indicated at 52′. The length adjustment mechanism typically is an adjustment screw coupled to second wire end 46′ as is known in the art for adjusting the free length of wire member 40. It should be appreciated that the system can also use a combination of the dynamic wire adjustment mechanism and the sire length adjustment mechanism.

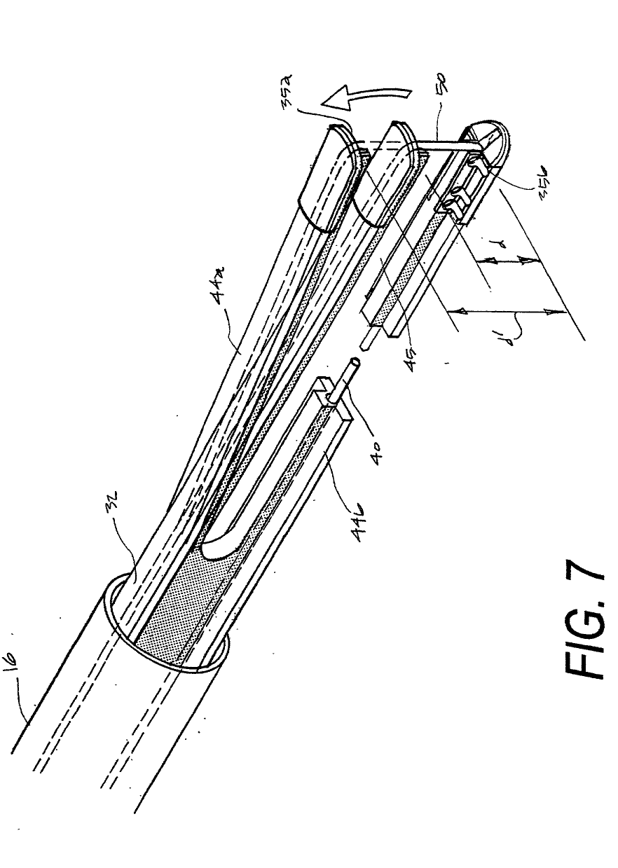

From an understanding of these wire length adjustment mechanisms, FIG. 7 shows a perspective view of the working end 5, again with the actual jaw members 8A and 8B not shown. In FIG. 7, the dimension between the first and second cam surfaces 35a and 35b, and the jaw's tissue-engaging faces 12A and 12B, can adjust under loads between transverse dimension d and transverse dimension d′ wherein the upper extension arm 44a flexes. The adjustment from transverse dimension d to dimension d′ can be under dynamic loads or in accordance with a pre-set wire length as described above. It should be appreciated that the amount of flexing of upper extension arm 44a in FIG. 7 may be exaggerated to for purposes of clarity. b. Electrode configuration for increased surface contact of engaged tissue volumes. As will be described below, the upper and lower jaw faces 12A and 12B carry upper and lower jaw electrodes 55 and 56, respectively, that are coupled to electrical source 60. As can be seen FIGS. 2 & 3, electrode 55 in the upper jaw comprises spaced apart left-side and right-side portions 55a and 55b that are coupled to electrical source 60 by separate leads 57a and 57b for reasons described below. Likewise, lower jaw electrode 56 has left- and right-side portions 56a and 56b that are coupled to electrical source 60 by separate leads 58a and 58b.

Referring now to FIG. 6, it can be understood that movement of the axially extendable member 32 to the second extended position relative to introducer member 16 for applying high compression forces to the jaws also causes the wire element portion 50 to pass through the captured tissue. Thus, the wire element 40 is coupled to the electrical source 60 by lead wire 64 to function as a cutting electrode as is known in the art.

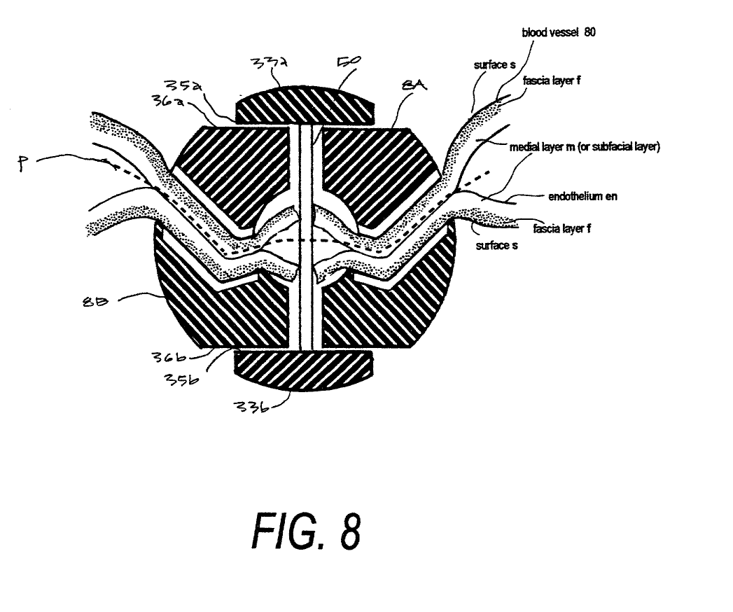

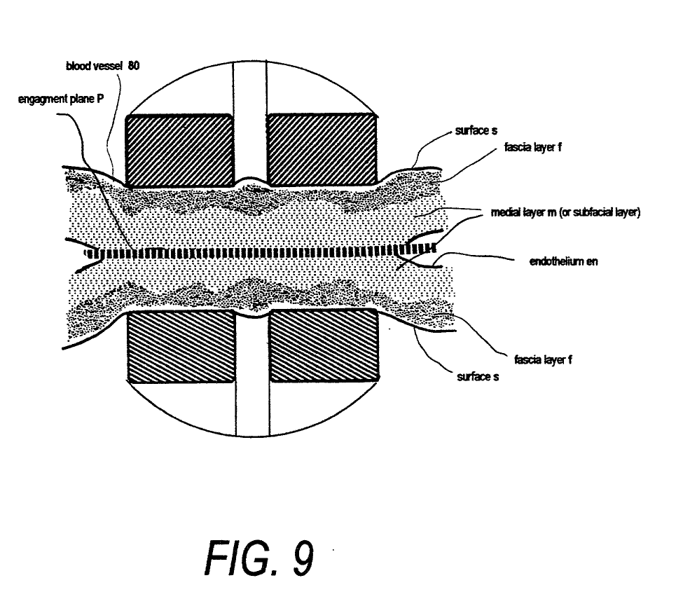

Now turning back to FIGS. 2 & 3, the novel shapes and surfaces of electrodes 55 and 56 in tissue-engaging faces 12A and 12B of jaws 8A and 8B can be described. FIG. 3 shows that the paired jaws in the second closed position generally define, in transverse sectional, an undulating shape wherein upper jaw face 12A is received by a cooperating shape in the lower jaw face 12B. The cross-sectional view of FIG. 8 shows that tissue-engaging faces 12A and 12B of jaws 8A and 8B define an engagement plane p (dashed line) that represents the plane in which targeted tissue is captured or engaged between the jaws before and during Rf energy delivery. Of particular interest, the engagement plane p: (i) has a non-linear form or non-planar from transverse to central axis 15 of the jaw structure, and/or (ii) has no portions that comprise a radial r of the axis 15 of the jaws (see FIG. 9). In prior art jaws, such an engagement plane P of the jaws typically is linear and also comprises a radial of a central axis of the jaws as shown in FIG. 9. By the term radial, it is meant that a radial line or plane is orthogonal to the central axis of the jaws and also be termed a radius (see FIG. 9). As can be seen in FIG. 9, such a radial r thus defines the shortest possible distance from central axis 15 to an exterior surface or edge of the jaw structure.

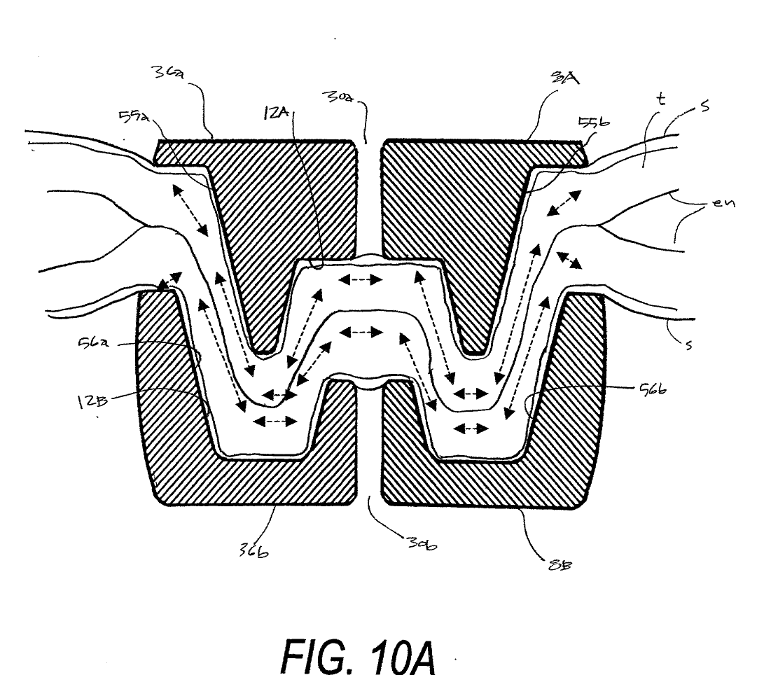

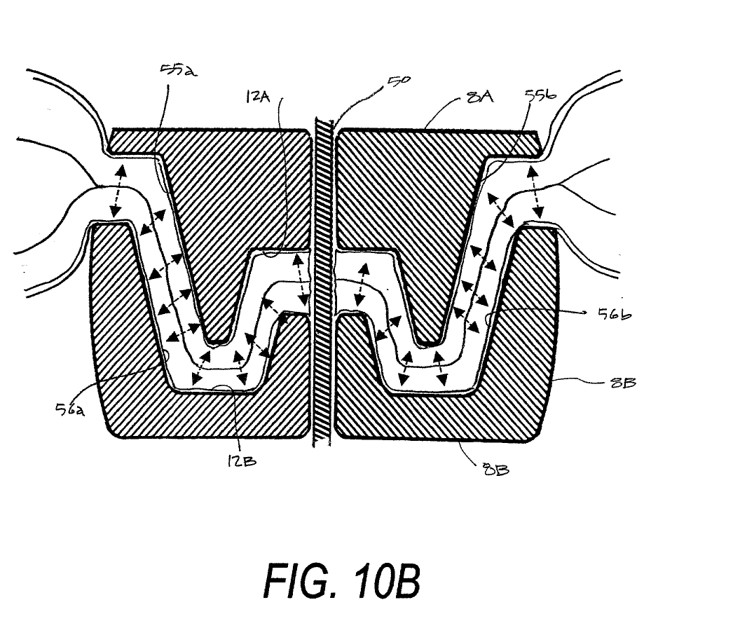

In the present invention, it has been found that tissue welds created by ohmic resistance to current flow between paired electrodes can be substantially enhanced by increasing the electrode surface areas engaging the tissue between upper and lower jaws 8A and 8B. The novel manner of providing such increased electrode engagement area, within a small diameter jaw form, is to not provide an engagement plane p that is a simple radial r of central jaw axis. Rather, the preferred embodiment of the invention provides non-radial forms for such an engagement plane p of FIG. 8. As can be seen in FIG. 8, a preferred engagement plane p is provided that extends at angles to a radial r thereby providing an increased dimension across the jaw faces 12A and 12B that carry the surfaces of electrodes 55 and 56. While FIG. 8 depicts one embodiment of an engagement plane p for accomplishing the method of the invention, FIG. 10 depicts a more preferred engagement plane p wherein jaw faces 12A and 2B have a deeply undulating form-and thus is still further removed from a radial form as shown in the prior art jaw cross-section of FIG. 9. The jaws shown in FIG. 10, for convenience, shows that the entire jaw members 8A and 8B comprise the electrode surfaces 55 and 56.

The electrodes 55 and 56 of any embodiment are of any suitable material such as aluminum, stainless steel, nickel titanium, platinum, gold, or copper. Each electrode surface preferably has a micro-texture (e.g., tiny serrations or surface asperities, etc.) for better engaging tissue and for delivering high Rf energy densities in engaged tissues as is known in the art. The bi-polar Rf current may be switched on and off by a foot pedal or any other suitable means such as a switch in handle (not shown).

Another embodiment of the invention (not shown) includes a sensor array of individual sensors (or a single sensor) carried in any part of the jaw assembly that is in contact with the tissue targeted for welding. Such sensors preferably are located slightly spaced apart from electrodes 55 and 56 for the purpose of measuring temperatures of tissue adjacent to the electrodes during a welding procedure. It should be appreciated however that the sensors also can measure temperature at the electrodes. The sensor array typically will consist of thermocouples or thermistors (temperature sensors that have resistances that vary with the temperature level). Thermocouples typically consist of paired dissimilar metals such as copper and constantan which form a T-type thermocouple as is known in the art. Such a sensor system can be linked to feedback circuitry that together with a power controller can control Rf energy delivery during a tissue welding procedure. The feedback circuitry can measure temperatures at one or more sensor locations, or sensors can measure the impedance of tissue, or voltage across the tissue, that is engaged between the transecting member and a jaw. The power controller then can modulate Rf delivery in order to achieve (or maintain) a particular parameter such as a particular temperature in tissue, an average of temperatures measured among multiple sensors, a temperature profile (change in energy delivery over time), a particular impedance level or range, or a voltage level as is known in the art.

Operation and use of the working end 5 of FIGS. 2-3 in performing a method of the invention can be briefly described as follows. FIGS. 10A-10B show a targeted tissue volume t that is captured between first and second jaws 8A and 8B. The targeted tissue t may be any soft tissue or anatomic structure of a patient's body and FIGS. 10A-10B depict a large diameter blood vessel 80 with vessel walls 82 having fascia layers indicated at f underlying exterior surfaces s, medial tissue layers m and endothelial layers en. In using the working end 5 to grasp, dissect, adjust and otherwise manipulate tissue before welding the tissue, the physician uses the first jaw-actuation mechanism described above, also described as the lower-compression mechanism that rotates the upper jaw with the sliding rods 24a and 24b coupled to the arcuate slots 22a and 22b.

In a first mode of operation for welding or cauterizing tissue, the physician again uses only the first jaw-actuation mechanism describe just above to clamp the tissue in tissue engagement plane p between jaws 8A and 8B. As described previously, the tissue compression is adequate for sealing thin tissue.

Of particular interest, as shown schematically in FIGS. 10A-10B, the tissue is welded or sealed by providing bi-polar current flow among the electrodes 55a-55b and 56a-56b in the paired jaws. The electrodes all are coupled to electrical generator 60 and controller 70 by independent leads that allow for rapid switching of polarities among pairs of electrodes to cause ohmic heating of tissue along different vectors.

FIG. 10A thus shows a first manner of vectoring bi-polar Rf current (indicated by arrows) wherein current flow is between the left-side electrodes (55a, 56a) and right-side electrodes (55b, 56b) to thereby cause current flow generally longitudinally within the blood vessel. In other words, the current is vectored to parallel to tissue engagement plane p. It has been found that such longitudinal current flow, together with the expanded surface areas of the electrodes provided by the undulating jaw faces, provide for even heating of captured tissue volumes with a lessened possibility of desiccating tissue.

FIG. 10B graphically depicts a second manner of vectoring bi-polar Rf current (indicated by arrows) wherein current flows in vectors generally orthogonal to tissue engagement plane p and between the upper jaw electrodes (55a, 55b) and lower jaw electrodes (56a, 56b). It has been found that such longitudinal current flow, together with the expanded electrode surface areas, allows for very rapid heating of the captured tissue volume.

In a preferred mode of operation, the controller 70 very rapidly switches the current flow between being (i) parallel to the tissue-engagement plane p, and (ii) orthogonal to the engagement plane p. In a more preferred mode of operation, the sensor circuitry described above is use to control energy delivery to the electrodes, and between parallel and orthogonal current flow relative to the engagement plane.

The above method of delivering Rf energy is adequate for many tissue sealing procedures. To fully insure that a thick or irregular tissue is welded, for example when sealing and transecting a blood vessel, the second high compression jaw closure mechanism is used. In other words, the axially extendable member 32 is moved from its rearward (first) position to its extended (second) position as shown in FIGS. 5 & 6 to apply very high compressive forces to the captured tissue. In this manner of operation, the wire member 40 operates at high Rf intensities suitable for cutting through the captured tissue as is known in the art. For example, for tissue cutting purposes, Rf frequencies may range from 500 kHz to 2.5 MHz, with power levels ranging from about 50 W. to 750 W., and open circuit voltages ranging as high as 9 Kv. In the configuration of FIGS. 2-6, the electrode wire member 40 is carried in an insulated extension member to insure that it does not contact paired electrodes 55 and 56.

After the captured tissue is transected, the paired cam surfaces 35a and 35b will control the transverse dimension d between the jaw faces an can apply tremendous compressive forces on the captured tissue. It has been found that such high compression assists in creating an effective weld in tissue. It is believed that the denaturation of proteins while under high compression allows for more complete intermixing of tissue constituents which then provides effective tissue fusion or sealing as the damaged tissue heals.

In delivering Rf energy for this phase of tissue welding, the current flow is obviously delivered orthogonal to the tissue engagement plane between upper jaw 8A and lower jaw 8B, since the tissue is transected. The controller 70 preferably has circuitry linked to electrical contacts in the introducer 16 and extendable member 32 to signal the position of the extendable member. Thus, when the extendable member 32 is moved to the second extended position, the controller would provide opposing polarities only in the upper jaw electrode 55 and lower jaw electrode 56 (and not side-to-side in the electrodes).

It has been found that a very effective weld w can be created within the transected ends of the vessel captured within the engagement plane p by the above method. The, the sectional illustration of FIG. 10B shows that a weld w can be created where the proteins (including collagen) are denatured, intermixed under high compressive forces, and then permanently fused upon cooling to seal or weld the margin of the transected vessel. Further, it is has been found believed that the desired weld effects can be accomplished substantially without collateral thermal damage to adjacent tissues indicates at 76 in FIG. 10B.

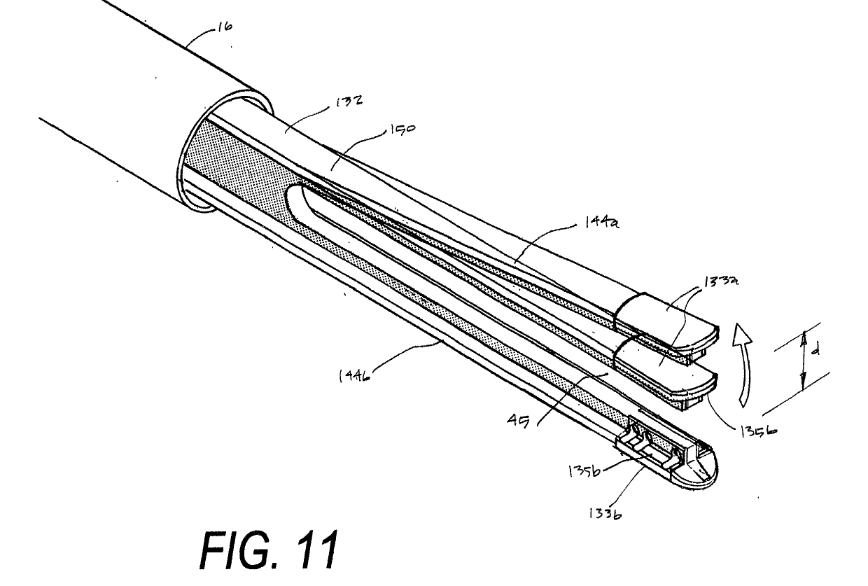

2. Type “B” Electrosurgical Jaw Structure for Sealing or Welding Tissue. Referring to FIG. 11, the working end 105 of Type “B” device is shown. More particularly, an alternative embodiment of axial-extending member is 132 is shown in FIG. 11. The member 132 carries first and second cam surfaces 135a and 135b at the distal ends of extension arms 144a and 144b. This embodiment is used with the upper and lower jaws 8A and 8B as depicted in FIGS. 2 & 3. This embodiment differs from the Type “A” embodiment in that the transverse dimension d across the free space 45 between the jaws faces 12A and 12B (see FIGS. 2 & 3) or between the first and second cam surfaces 135a and 135b is not mechanically adjusted by a transverse member. Rather, the space 45 between the jaws faces 12A and 12B is dynamically adjustable by the spring constant (flexibility) of the material comprising the axial-extending member is 132. The material of the axial-extending member is 132 is a springable metal, plastic or combination thereof that provides a unitary hinge point indicated at 150 where the upper extension arm 144a flexes to thereby allow the jaws to flex apart as indicated by the arrow in FIG. 11. The type of jaw closing mechanism is not described as a high compression jaw closure mechanism. Rather, this type of jaw closing mechanism is useful for less elongate jaw length wherein the objective of the invention is to provide for parallel jaw surfaces 12A and 12B no matter the thickness of captured tissue to optimized Rf energy delivery. This embodiment of extension member 132, when combined with the floating pivot mechanism shown in FIG. 4, will allow for substantially parallel jaws engagement about an engagement plane p when the spring forces of hinge point 150 and the floating pivot are properly balanced.

While FIG. 11 shows a unitary hinge point 150 formed into the material of member 132, it should be appreciated that a pin-type hinge with any sort of spring also may comprise hinge point 150 and fall within the scope of the invention. For convenience, a wire or blade member for transecting tissue is not shown in FIG. 11. However, it is obvious that any type of fixed or reciprocating blade or electrode may be added to the axial-extending member of FIG. 11A.

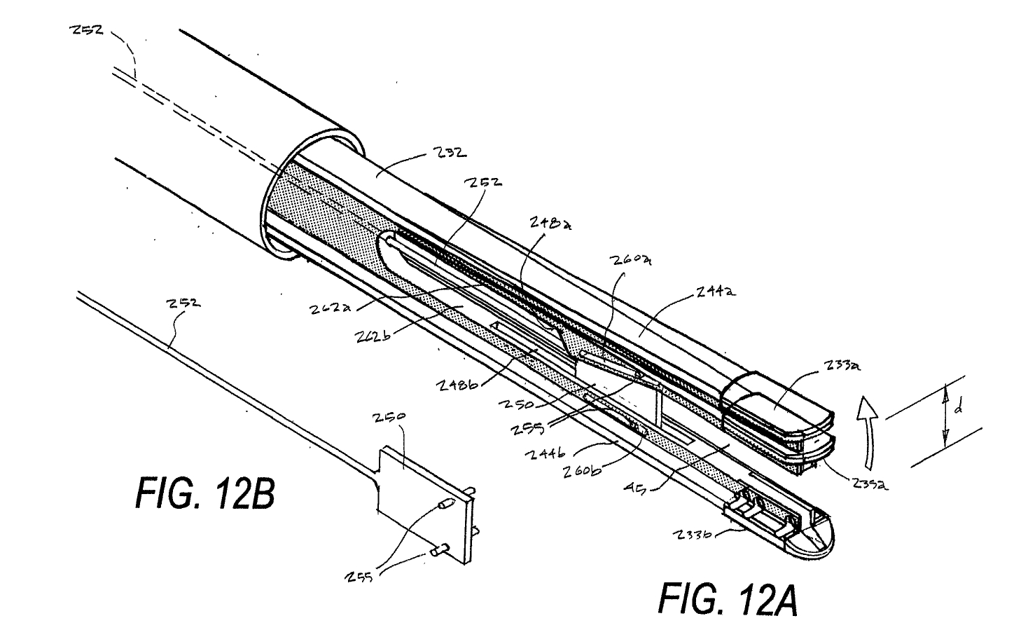

3. Type “C” Electrosurgical Jaw Structure for Welding Tissue. The functional component of a Type “C” working end 205 is shown in FIGS. 12A & 12B that is adapted to cooperate with jaws 8A and 8B as shown in FIGS. 2 & 3. In the Type “C” system of FIG. 12A, the elements that are identical to those of the Type “A” embodiment have the same reference numeral+200. FIG. 12A shows an alternative embodiment of axial-extending member is 232 in which first and second cam surfaces 235a and 235b are carried at distal ends of extension arms 244a and 244b. In this embodiment, the transverse dimension d across the free space 45 between jaws faces 12A and 12B (see FIGS. 2 & 3) and between first and second cam surfaces 235a and 235b can be adjusted to any selected dimension from a handle portion of the instrument (not shown). A mechanism that is transverse to the extension arms 244a and 244b is provided to adjust the transverse dimension. More in particular, this embodiment has axial slot portions 248a and 248b in extension arms 244a and 244b that receives a slidable transverse member 250 that carries engagement pins 255 extending therethrough. The transverse member 250 has a proximal potion 252 that extends to the instrument handle wherein any sort of locking mechanism can maintain the transverse member 250 in a fixed position relative to the axial-extending member is 232. FIG. 12B shows a view of the transverse member 250 and engagement pins 255 de-mated from the axial-extending member is 232 for clarity. It can be seen that cooperatively angled slots 260a and 260b are provided in web portions 262a and 262b of the extension arms 244a and 244b. It can easily be understood that by axial slidable movement of transverse member 250, the engagement pins 255 engage slots 260a and 260b to move the extension arms 244a and 244b closer together or further apart-in other words adjusting the transverse dimension d between the jaws faces in a closed position. This type of jaw closing mechanism thus provides a high compression jaw closure mechanism for applying very high pressures on captured tissue, which is similar to the effect provided by the Type “A” system above. This embodiment of extension member 132 preferably is combined with the floating pivot mechanism shown in FIG. 4 to for substantially parallel engagement of the jaws about an engagement plane p, as described above. FIG. 12, for convenience, does not show a wire or blade member for transecting tissue. However, it is obvious that any type of fixed or reciprocating blade or electrode may be added to the axial-extending member 232 of FIG. 12.

The mechanism for adjusting the transverse dimension between the cam surfaces also can include any type of leaf spring (not shown) coupling medial or distal portions of extension arms 144a and 144b of FIG. 11 for controlling the compression applied to the jaw surfaces. The mechanism for adjusting the transverse dimension between the cam surfaces also can include any type of lever arm(s) (not shown) that couple medial or distal portions of extension arms 144a and 144b of FIG. 11 that are adjustable by an adjustment member extending to the handle portion, for example an adjustable scissor-jack mechanism known in the art.

It should be appreciated that a Type “D” working end (not shown) can comprise any working end described above in combination with an additional translatable central electrode as disclosed in co-pending U.S. patent application Ser. No. ______ filed Oct. 23, 2000 (Docket No. SRX-001) titled Electrosurgical Systems and Techniques for Sealing Tissue (incorporated herein by reference). The jaw structure described above also can include semiconductor cooling elements to cool tissue volumes collateral to the tissue engaged by jaw members as was first disclosed by an author in co-pending U.S. patent application Ser. No. 09/110,065 filed Jul. 3, 1998, which is incorporated herein by this reference.

Although particular embodiments of the present invention have been described above in detail, it will be understood that this description is merely for purposes of illustration. Specific features of the invention are shown in some drawings and not in others, and this is for convenience only and any feature may be combined with another in accordance with the invention. Further variations will be apparent to one skilled in the art in light of this disclosure and are intended to fall within the scope of the appended claims.

What is claimed is:

1. An electrosurgical working end for applying controlled pressure on captured tissue, comprising:
an introducer member coupled to a working end that carried paired jaws actuatable to open and close relative to a tissue engagement plane;
an axially-extendable member with first and second cam surfaces that define a transverse dimension therebetween, the first and second cam surfaces slidably engaging cooperating surfaces of said first and second jaws to move the jaws toward and away from the tissue engagement plane; and
a transverse member coupling the first and second cam surfaces, said transverse member allowing said transverse dimension between the first and second cam surfaces to vary thereby providing greater or lesser compressive forces on captured tissue about the tissue-engaging plane.

2. The electrosurgical instrument of claim 1 wherein the axially-extendable carries said first and second cam surfaces at distal end of spaced apart extension arms.

3. The electrosurgical instrument of claim 1 wherein the transverse member comprises a wire member.

4. The electrosurgical instrument of claim 2 wherein the wire member provides a variable transverse dimension between the first and second cam surfaces by coupling to a spring.

5. The electrosurgical instrument of claim 2 wherein said wire member is translatable between said first and second cam surfaces.

6. The electrosurgical instrument of claim 5 wherein the translatable wire member is substantially flexible.

7. The electrosurgical instrument of claim 4 wherein the wire member is coupled to an electrical source thereby functioning as an electrode.

8. The electrosurgical instrument of claim 1 wherein the transverse member comprises an actuatable member that adjustably couples the extension arms.

9. An electrosurgical working end for applying controlled pressure on captured tissue, comprising:
a body member carrying first and second jaw members actuatable to open and close about a tissue engagement plane;
a translatable extension member that is coupled to first and second spaced apart extension arms, the arms carrying a first and second cam surfaces for engaging cooperating surfaces of said first and second jaw members to actuate the jaws;
wherein said first and second cam surfaces define a space therebetween that defines a transverse dimension that is not fixed.

10. The electrosurgical instrument of claim 9 wherein the first and second spaced apart extension arms are of a spring-type material thereby providing adjustment to said transverse dimension.

11. The electrosurgical instrument of claim 9 wherein a medial portion of the first and second spaced apart extension arms are coupled by a spring member.

12. The electrosurgical instrument of claim 9 wherein a medial portion of the first and second spaced apart extension arms are coupled by a transverse slidable member.

13. The electrosurgical instrument of claim 9 wherein a medial portion of the first and second spaced apart extension arms are coupled by a transverse pivotable member.

14. An electrosurgical method for sealing tissue, comprising the steps of:
providing a working end carried at the distal end of an introducer member that has paired jaws actuatable to close toward a tissue engagement plane;
capturing a tissue volume within the tissue engagement plane;
causing Rf current flow within the tissue volume substantially parallel to the engagement plane from spaced apart electrodes in left and right sides of the paired jaws; and
causing Rf current flow within the tissue volume substantially orthogonal to the engagement plane from electrodes in each jaw.
